(19) United States
(12) Patent Application Publication
(10) Pub. No.: US 2026/0367061 A1
(43) Pub. Date: Nov. 19, 2026

(54) GEOTHERMOMETRIC DETECTION OF CONTAMINANT FLUIDS IN OIL AND GAS WELLS

(71) Applicant: SAUDI ARABIAN OIL COMPANY, Dhahran (SA)

(72) Inventor: Peter BIRKLE, Dhahran (SA)

(73) Assignee: SAUDI ARABIAN OIL COMPANY, Dhahran (SA)

(21) Appl. No.: 19/209,601

(22) Filed: May 15, 2025

(51) Int. Cl. G01N 33/18; E21B 43/20; E21B 47/07; E21B 49/08; G01V 9/00

(52) U.S. Cl. CPC G01N 33/18; E21B 47/07; E21B 49/081; G01V 9/005; E21B 43/20

(57) ABSTRACT

A computer-implemented method for identifying contamination in formation water includes extracting a fluid sample of formation water from a hydrocarbon well, performing geochemical analysis on the fluid sample to determine chemical composition of the formation water, and generating a calculated temperature using a geothermometer method and the chemical composition of the formation water. The computer-implemented method further includes performing temperature logging in the well at one or more depths to obtain a measured temperature, comparing the calculated temperature to the measured temperature to determine a presence of contamination within the formation water, and modifying hydrocarbon extraction operations in response to the presence of contamination within the formation water.

### FIELD OF THE DISCLOSURE

The present disclosure relates generally to the identification of contamination within produced water from hydrocarbon wells.

### BACKGROUND OF THE DISCLOSURE

During drilling and extraction operations in hydrocarbon wells, primary formation water is commonly recovered from downhole and can impact reservoir conditions and technical decisions for the operation of hydrocarbon wells. For example, knowing the geochemical composition of pristine formation water, drilling mud can be specifically formulated to counterbalance the formation fluid pressure from the primary formation water. The contamination of the primary formation water can alter the operating conditions of the hydrocarbon wells and hydrodynamic and thermal properties of the primary formation water. Further, the presence of contaminated primary formation water can signal the presence of leaking equipment or wellbore casing.

As such, efforts have been made to determine the presence and severity of contamination in water produced from wellbore operations. In some applications, environmental tracers naturally existing within the formation or artificial tracers (e.g., uranine, fluorescein, KBr, KI) introduced into the wellbore and formation can be tracked over time to determine contamination. Fluorescent dye tracers like fluorescein and rhodamine WT have been used as contamination markers to allow for calculations of the chemical and isotopic composition of formation water. In applications using oil-based drilling mud, the produced water can be analyzed for organic carbon components (C5 to C36), and geochemical fingerprints can be compared to reference drilling mud to determine contamination levels. In further applications, oil-based mud contamination levels can be assessed based upon a comparison of an oil/gas ratio for fluids native to the formation against an apparent gas/oil ratio for a contaminated fluid. In further applications still, chloride concentrations of produced water are routinely measured at the wellhead during drilling operations in oil and gas wells to determine fluctuations in the composition of produced water. Due to these natural fluctuations in the geochemical composition of formation water and potential mixtures with residuals from ascending drilling fluids, measured Cl concentrations are not deterministic for quantifying reservoir fluid properties. Especially when using water-based mud as drilling fluid, formation water and contaminant fluids (drilling or well stimulation fluids) cannot be quantitatively differentiated by chloride concentration measurements alone. Preserved downhole samples are recovered to determine the representative fluid composition at a specific depth and reservoir unit. However, each of these contamination identification methods can fail to be deterministic of actual fluid parameters for the produced water, can be costly and time-consuming, and the reliability of these methods can fail to yield useful data.

Accordingly, systems and methods for identifying and quantifying contamination levels of produced water in hydrocarbon wells are desirable to provide accurate formation water properties and to enhance hydrocarbon production using the fluid properties.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

In an embodiment consistent with the present disclosure, a computer-implemented method for identifying contamination in formation water includes extracting a fluid sample of formation water from a hydrocarbon well, performing geochemical analysis on the fluid sample to determine chemical composition of the formation water, and generating a calculated temperature using a geothermometer method and the chemical composition of the formation water. The computer-implemented method further includes performing temperature logging in the well at one or more depths to obtain a measured temperature, comparing the calculated temperature to the measured temperature to determine a presence of contamination within the formation water, and modifying hydrocarbon extraction operations in response to the presence of contamination within the formation water.

In another embodiment, a system for identifying contamination in formation water includes a fluid sampler included at or near a wellbore of interest, the fluid sampler operable to extract a sample of formation water from a depth of the wellbore, a well logging tool included at or near the wellbore and operable to determine a measured temperature within the wellbore at the depth, and a fluid contamination determination engine in communication with the fluid sampler and the well logging tool and operable to detect contamination within the sample and the wellbore. The fluid contamination determination engine is configured for implementation on at least one processor and includes a geochemical analysis module operable to receive the sample of the formation water and perform a geochemical analysis extracting cationic component concentrations of the sample, a geothermometric calculation module operable to utilize the cationic component concentrations of the sample to generate a calculated temperature via one or more geothermometer methods, and a fluid contamination assessment module operable to compare the measured temperature and the calculated temperature and determine a presence of contamination within the sample of formation water.

In a further embodiment, a non-transitory computer-readable medium stores machine-readable instructions, which, when executed by a processor of an electronic device, cause the electronic device to receive a sample of formation water for a wellbore of interest from a fluid sampler in communication with the electronic device, perform geochemical analysis on the sample to determine cationic component concentrations in the sample, generate a calculated temperature for the sample using the cationic component concentrations and one or more geothermometer methods, receive a measured temperature for the wellbore from a well logging tool in communication with the electronic device, and compare the calculated temperature to the measured temperature to determine a presence of contamination within the formation water of the wellbore.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is a schematic view of an example system for identifying fluid contamination in a hydrocarbon well and modifying hydrocarbon extraction operations in response to the fluid contamination, according to an embodiment of the present disclosure.

FIG. 2 is a schematic view of an example electronic device operable to identify fluid contamination and adjust hydrocarbon extraction at the hydrocarbon well, according to an embodiment of the present disclosure.

FIG. 3 is a schematic view of an example workflow for assessing fluid contamination in a hydrocarbon well and using the assessment in operational applications therein, according to an embodiment of the present disclosure.

FIG. 4 illustrates a method for identifying fluid contamination in a hydrocarbon well and modifying hydrocarbon extraction operations in response to the fluid contamination, according to one or more embodiments of the present disclosure.

FIG. 5A depicts vertical trends of geothermal gradients and measured reservoir temperatures at specific depths compared with geothermometer temperatures from produced water.

FIG. 5B depicts the discrepancies between calculated reservoir temperatures and measured reservoir temperatures.

FIG. 6 illustrates one example of a computer system that can be employed to execute one or more embodiments of the present disclosure.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally to the identification of contamination within produced water from hydrocarbon wells, and, more particularly, to utilizing geothermometric calculations and well logging temperatures to determine contamination through temperature discrepancies. Embodiments disclosed herein include systems and methods for the sampling, analysis, and utilization of formation water samples from a wellbore of interest. The formation water samples can enable the determination of geothermometer temperatures to be compared against the temperatures measured through well logging. The systems and methods disclosed herein are operable to identify contamination within the formation water based upon discrepancies between the measured and calculated temperature, suggesting that the formation water has been disturbed with drilling fluids or other contaminants.

The methods and systems disclosed herein can further include providing adjustments to the operational parameters of the wellbore and hydrocarbon extraction equipment therein, based upon the contamination of the formation water. The adjustments can include modified drilling operations, altered estimates of hydrocarbon-in-place values, tracking of processes for enhanced oil recovery, among others. The combined assessment of contaminants within the formation water and the rectification of issues or adjustment of operating parameters can enable rapid, reliable, and widely-applicable solutions for formation water contamination. The disclosed methods and systems can utilize simple samples and logging tools to determine contamination within the wellbore of interest without introducing additional tracers, organic fingerprinting, or other costly and time-consuming monitoring techniques.

The detection of contaminated fluids in produced water with geothermometers can represent a decision-taking tool for the modification of drilling mud properties during drilling operation, the detection for the source of casing failure, enhanced hydrocarbon-in-place calculations by precise water resistivity values, and EOR-related tracking of the arrival of injected seawater in the production zone. The quantitative detection of drilling fluid contamination can decrease the risk of stuck holes and the loss of rig time, and can be utilized to adjust and improve several drilling parameters such as bit penetration rate, drill hole cleanliness, well hydraulics, filter cake formation and drilling fluid stability.

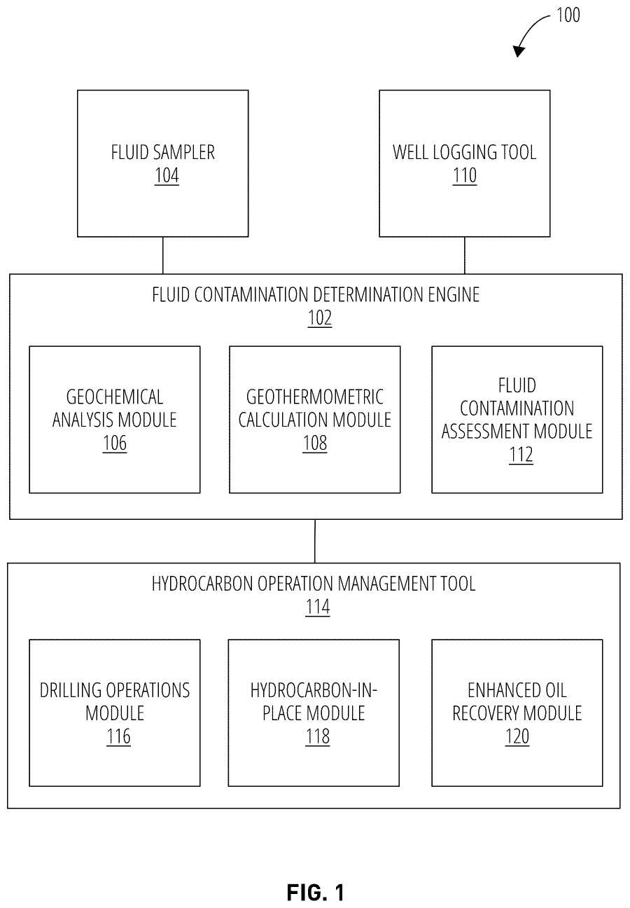

FIG. 1 is a schematic view of an example system 100 for identifying fluid contamination in a hydrocarbon well and modifying hydrocarbon extraction operations in response to the fluid contamination, according to an embodiment of the present disclosure. System 100 includes a fluid contamination determination engine 102 operable to receive formation water samples from a fluid sampler 104. Fluid contamination determination engine 102 can include and utilize a geochemical analysis module 106 for extracting cationic component concentrations to be used in a geothermometric calculation module 108. Geothermometric calculation module 108 can utilize these concentrations to generate a calculated temperature of the wellbore to be compared to a measured temperature of a well logging tool 110. Using the measured and calculated temperatures, a fluid contamination assessment module 112 of fluid contamination determination engine 102 can identify contamination within the formation water based on discrepancies between the temperatures. After identification the presence of contaminated or pure formation water, a hydrocarbon operation management tool 114 can enable modified hydrocarbon operations to capitalize on pure formation water, or to correct for contaminated formation water. Hydrocarbon operation management tool 114 can include a drilling operations module 116, a hydrocarbon-in-place module 118, and an enhanced oil recovery module 120, each module operable to adjust one or more parameters of hydrocarbon operations within the well in response to the identified contamination levels of the formation water.

In embodiments, fluid contamination determination engine 102 and hydrocarbon operation management tool 114 may be implemented on one or more computing devices at the same or different locations. Fluid contamination determination engine 102 including geochemical analysis module 106, geothermometric calculation module 108, and fluid contamination assessment module 112 may be implemented in software, firmware, hardware or any combination thereof for execution on the same or different processors. Hydrocarbon operation management tool 114 including drilling operations module 116, hydrocarbon-in-place module 118, and enhanced oil recovery module 120 may also be implemented in software, firmware, hardware or any combination thereof for execution on the same or different processors.

Fluid contamination determination engine 102 can be communicatively coupled to the fluid sampler 104 and the well logging tool 110, and can be operable to detect contamination within a fluid sample obtained from fluid sampler 104 and the wellbore. Fluid sampler 104 can be included at or near a wellbore of interest, and can be operable to extract a sample of formation water from a specific depth of the wellbore. Fluid sampler 104 can retrieve produced water samples from surface outlets of the wellbore, or can include bottomhole tools operable to extract fluids from the wellbore of interest.

The samples retrieved via fluid sampler 104 can be received within fluid contamination determination engine 102, and provided to geochemical analysis module 106 therein. In some embodiments, these samples are filtered (e.g., using a 0.45 μm pore-size filter) and acidified to a pH below 2.0 for cation and metal analysis by geochemical analysis module 106. Geochemical analysis module 106 can be operable to receive the sample of the produced water and perform a geochemical analysis to extract cationic component concentrations of the sample. In some embodiments, the cationic component concentrations can include the concentration of silica and the proportions of sodium, potassium, lithium, calcium, and magnesium in the sample within geochemical analysis module 106. These cationic component concentrations can be utilized for the generation of calculated temperature via geothermometry and geothermometric calculation module 108.

Geothermometric calculation module 108 can, accordingly, be operable to utilize the cationic component concentrations of the sample to generate a calculated subsurface temperature via one or more geothermometer methods, under the assumption that the abundance and proportion of alkali metals and alkaline-earth minerals in the fluid phase are directly linked to specific thermal reservoir conditions. Although initially developed for geothermal reservoirs, the Mg—Li and Na—K—Ca geothermometers (with Mg correction) have proven effective for oil-field water. The geothermometer methods employed by geothermometric calculation module 108 can include a magnesium-corrected sodium-potassium-calcium geothermometer, a magnesium lithium geothermometer, a sodium-lithium geothermometer, or a combination thereof.

In some embodiments, geothermometric calculation module 108 can employ the below equation to obtain the temperature for the Na—K—Ca geothermometer:

$${t}_{Na - K - Ca} [ °C ] = \frac{1647}{log ( \frac{Na}{K} ) + β [ log ( \frac{\sqrt{Ca}}{Na} ) + 2.06 ] + 2.47} - 273$$

In these embodiments, the selected β value can depend on the reservoir temperature based upon the below equation:

$$β = \frac{4}{3} for t < 100 °C ; = \frac{1}{3} for t > 100 °C$$

The Mg-corrected Na—K—Ca geothermometer is based on the temperature estimated from the Na—K—Ca geothermometer above, as well as a variable R, which is derived through the below equation:

$$R = \frac{{C}_{Mg}}{{C}_{Mg} + 0.61 {C}_{Ca} + 0.31 {C}_{K}} \times 100$$

where concentrations of Mg (C_{Mg}), K (C_{K}), and Ca (C_{Ca}) in are expressed in mg/l. For R values from 5 to 50, the correction is given by the below equation:

$$Δ {t}_{Mg} = 10.66 - 4.7415 R + 325.87 {( log R )}^{2} - 1.032 \times {10}^{3} ( log \frac{{R )}^{2}}{T} - 1.968 \times {10}^{7} ( \frac{{log R )}^{2}}{{T}^{2}} + 1.605 \times {10}^{7} ( log \frac{{R )}^{3}}{{T}^{2}}$$

where Δt_{Mg} (for 5<R<50) is the temperature correction in degrees Celsius being subtracted from the Na—K—Ca temperature and T is the temperature in Kelvin calculated from the Na—K—Ca geothermometer. The final Mg-corrected Na—K—Ca temperature (t_{Na—K—Ca Mg-corr}) can be obtained using the below equation:

t_{Na—K—Ca Mg corr}=t_{Na—K—Ca}−Δt_{Mg}

In some embodiments, temperatures from Mg—Li and Na—Li geothermometers are calculated as in the first and second equations below, respectively:

$${t}_{Mg - Li} [ °C ] = \frac{2200}{log ( \sqrt{Mg / Li} ) + 5.47} - 273$$

$${t}_{Na - Li} [ °C ] = \frac{1590}{log ( \sqrt{Na / Li} ) + 0.779} - 273$$

Those having ordinary skill in the art will readily appreciate, however, that any accurate geothermometer method can be utilized by geothermometric calculation module 108 to generate a calculated temperature, without departing from the scope of this disclosure. For comparison to the calculated temperature from geothermometric calculation module 108, system 100 can include well logging tool 110 located at or near the wellbore and in communication with fluid contamination determination engine 102. Well logging tool 110 can be used during drilling of the wellbore to establish local and regional formation temperatures, as well as geothermal gradients throughout the depth of the wellbore. Geothermal gradients for reservoirs and wellbores are about 20 to 35 °C/km, though significantly higher values (up to 85 °C/km) can be found in tectonically active areas and lower values (0.05 °C/km) can be found in stable continental platforms. In some embodiments, fluid contamination determination engine 102 can receive recent logging data from well logging tool 110 that correlates to previously performed logging operations. In further embodiments, however, well logging tool 110 can be actively run at request of fluid contamination determination engine 102 to obtain updated logging data for temperature within the wellbore.

Using the calculated temperature of geothermometric calculation module 108 and the measured temperature from well logging tool 110, fluid contamination determination engine 102 can employ fluid contamination assessment module 112 to compare these temperatures and determine a presence of contamination within the sample of formation water (e.g., via hydrocarbons, corrosion inhibitors, drilling fluid components, stimulation fluids, and/or condensate water). As a basic assumption, identical values with maximum discrepancies of ±10% for the calculated geothermometer temperature and the measured logging temperature indicate the recovery of pristine formation water from the wellbore without the influence of non-reservoir fluids. Thus, if fluid contamination assessment module 112 identifies identical (within a prescribed margin of error) temperatures for the measured and calculated values, fluid contamination assessment module 112 can determine that no significant contamination is present within the formation.

In contrast, if fluid contamination assessment module 112 identifies significant discrepancies (outside of the margin of error) between the measured and calculated temperatures, fluid contamination assessment module 112 can determine that there is contamination within the produced water, such that drilling fluids and/or stimulation fluids from exploration or operation have entered the formation. Fluid contamination assessment module 112 can make this determination based upon the short residence time of drilling and/or stimulation fluids within the reservoir preventing the achievement of thermodynamic equilibrium conditions between mineral and solution or solute and solute in the formation. Therefore, the geochemical composition of affected produced water samples, obtained from geochemical analysis module 106, is not representative of pristine formation water composition, but rather consists of formation water with a varying mixing degree of contaminant fluids.

Fluid contamination determination engine 102 can communicate an identified contamination of formation water with hydrocarbon operation management tool 114 of system 100. Hydrocarbon operation management tool 114 can accordingly modify hydrocarbon operations within the wellbore based upon the presence or absence of contaminants in the formation water. Hydrocarbon operation management tool 114, and the operations thereof, can rely upon an understanding of the composition of pristine formation water and the presence of contaminant fluids in produced water for making informed technical decisions in drilling and production operations.

Thus, hydrocarbon operation management tool 114 can include drilling operations module 116, which can be operable to generate instructions for adjusting drilling fluid properties, correcting circulation loss within the wellbore, repairing casing failures within the wellbore, or a combination thereof. An accurate identification of salinity and density of the formation water can enable optimization of the mud weight of oil-based and water-based muds for use in drilling operations. As such, drilling operations module 116 can generate instructions or directly control mud weights of the drilling fluid based upon the communicated composition and contamination of the formation water, particularly based upon geochemical composition, density, and rheological differences between the drilling fluid and the formation water. Thus, adjustments to the drilling fluid can ensure a wellbore pressure greater than the formation pressure to avoid unstable borehole conditions caused by the inflow of formation water, and can further include adjusting the drilling fluid weight (e.g., via barite or hematite) to increase density thereof.

In further embodiments, drilling operations module 116 can be utilized in identifying lost circulation within the wellbore. Lost circulation, or the uncontrolled flow of drilling fluid into the formation, can be detected and corrected via the hydrocarbon operation management tool 114 through the levels of contamination detected within the formation water. Further, casing failure, including wellbore leakages, can be similarly identified via the levels of contamination entering the formation water. However, the location of the specific casing undergoing failure can utilize a plurality of measured and calculated temperatures through a depth of the wellbore to determine the zone of highest contamination, thus correlating to the casing interval undergoing failure.

In some embodiments, hydrocarbon operation management tool 114 can include hydrocarbon-in-place module 118, which is operable to generate an estimate of hydrocarbon-in-place levels within the wellbore and generate instructions to modify extraction operations using the estimate. In some embodiments, hydrocarbon-in-place module 118 can utilize the Archie equation to calculate the water saturation (Sw) of the uninvaded zone in a formation next to a borehole from well log parameters, and can be expressed as follows:

$${S}_{w}^{n} = \frac{{R}_{w}}{( {Φ}^{m} \times {R}_{t} )} ]$$

where S_{w} is water saturation of the uninvaded zone, n is saturation exponent, which varies from 1.8 to 4.0 but normally is 2.0, R_{w} is the formation water resistivity at formation temperature, φ is the porosity, m is the cementation exponent, which varies from 1.7 to 3.0 but normally is 2.0, Rt is the true resistivity of the formation, corrected for invasion, borehole, thin bed, and other effects.
R_{w} can be a critical parameter for hydrocarbon-in-place module 118, and is generally derived from calculated values from spontaneous potential (SP) log, estimated from R_{w} catalogs or from wet zone Ro value, and measured from water samples. In practice, the salinity of identified pristine formation water samples can be converted in R_{w} values and can be utilized as input parameter for the Archie equation to improve the calculation of S_{w} properties. Water saturation (S_{w}) is inversely correlated to hydrocarbon saturation (SH) as follows:

S_{H}=1−S_{w}

The resulting determination of S_{H} properties can be utilized for an improved hydrocarbon-in-place estimation. In contrast to drilling operations module 116, hydrocarbon-in-place module 118 can be utilized when fluid contamination assessment module 112 identifies pristine formation water from the samples of fluid sampler 104. In these embodiments, the fluid parameters of the pristine formation water can provide accurate estimations of hydrocarbon-in-place values, with high-confidence of uncontaminated fluid parameters being employed.

In further embodiments still, hydrocarbon operation management tool 114 can include enhanced oil recovery module 120, which is operable to monitor and control an injection of seawater in the wellbore for enhanced oil recovery based upon the presence of contamination within the sample of formation water. In these embodiments, constant or interval-based samples and logs can be obtained for the wellbore via system 100. Fluid contamination determination engine 102 can be utilized to monitor the propagation of injected seawater toward the production zones, such that an accurate time of arrival and level of contamination can be utilized in controlling the amount of seawater injected downhole. Homogeneous values for well log and geothermometer temperatures suggest the production of pristine formation water, while discrepancies indicate the arrival of seawater with its subsequent mixing or displacement of pristine formation water close to the borehole. Through the use of enhanced oil recovery module 120, the seawater propagation can be properly controlled to limit water cut and negative impacts to hydrocarbon production, while further providing timely verification of seawater injection. Thus, the various modules of hydrocarbon operation management tool 114 can utilize the detection of contamination to provide adjustment and improvement of several drilling parameters such as bit penetration rate, drill hole cleanliness, well hydraulics, filter cake formation and drilling fluid stability, while further decreasing the risk of stuck holes and the loss of rig time.

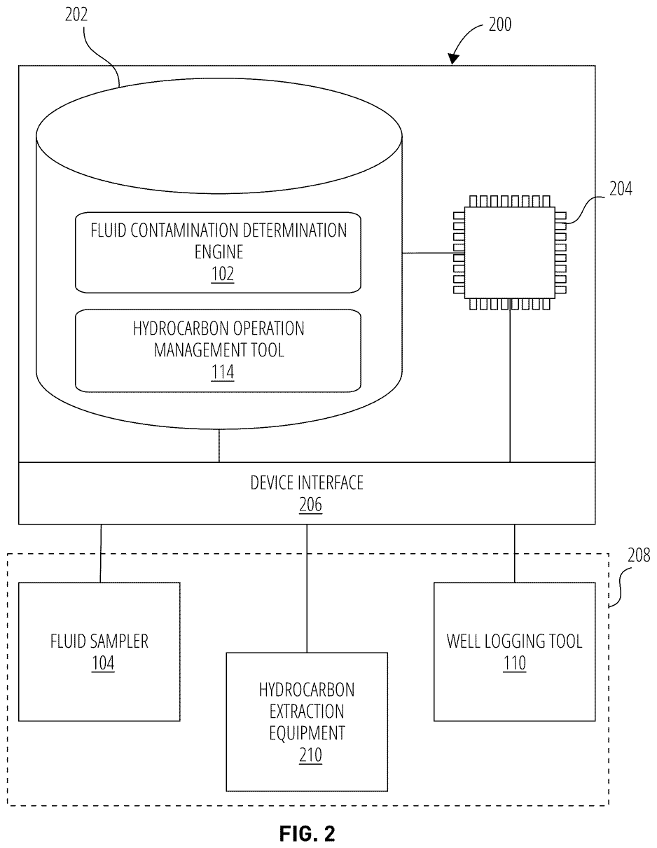

FIG. 2 is a schematic view of an example electronic device 200 operable to identify fluid contamination and adjust hydrocarbon extraction at the hydrocarbon well, according to an embodiment of the present disclosure. Electronic device 200 can primarily include a non-transitory computer-readable medium 202 communicatively coupled to a processor 204 for the execution of instructions stored within the non-transitory computer-readable medium 202. Electronic device 200 can further include device interface 206 operable to communicate with various communicatively-coupled tools located at or near hydrocarbon well 208. The tools communicatively coupled to electronic device 200 and device interface 206 can include hydrocarbon extraction equipment 210, fluid sampler 104, and well logging tool 110, each operable to send and receive data or instructions to and from electronic device 200.

As discussed above, non-transitory computer-readable medium 202 can be communicatively coupled to processor 204 as part of electronic device 200, such that any instructions stored within non-transitory computer-readable medium 202 can be executed via processor 204. In some embodiments, the instructions stores in non-transitory computer-readable medium 202 can include both fluid contamination determination engine 102 and hydrocarbon operation management tool 114. As such, electronic device 200 can be operable to identify contamination within formation water of hydrocarbon well 208, generate instructions to modify hydrocarbon extraction therein based upon the identified contamination, and transmit said instructions, via device interface 206, to hydrocarbon extraction equipment 210 within the wellbore for performance of these instructions.

In some embodiments, instructions generated within electronic device 200 can include an adjustment of drilling fluid composition, a repair of failed casing within the wellbore, a correction of a lost-circulation zone, or a combination thereof. In further embodiments, electronic device 200 can maintain communication with fluid sampler 104, well logging tool 110, and hydrocarbon extraction equipment 210, such that electronic device 200 can request a further sample of formation water from the fluid sampler and compare a calculated temperature of the further sample to the measured temperature to monitor for contamination. Thus, electronic device 200 can provide on-demand analysis of contamination within hydrocarbon well 208, as well as constant monitoring of contamination within hydrocarbon well 208, based upon the desired application.

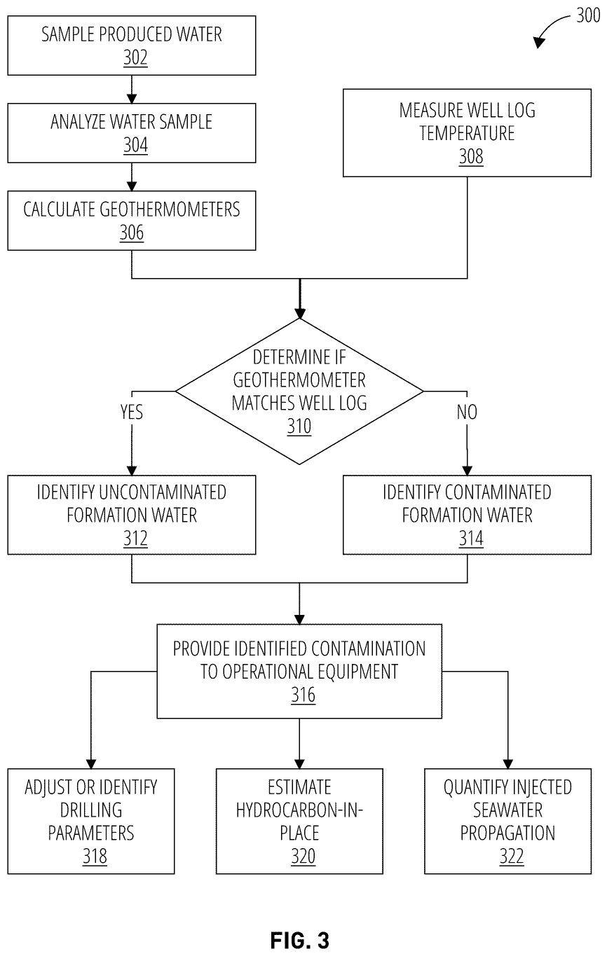

FIG. 3 is a schematic view of an example workflow 300 for assessing fluid contamination in a hydrocarbon well (e.g., hydrocarbon well 208) and using the assessment in operational applications therein, according to an embodiment of the present disclosure. Workflow 300 can begin at 302 with obtaining a sample of formation water from within the hydrocarbon well. The obtained sample can be analyzed at 304 to determine cationic component concentrations within the obtained sample of formation water. The cationic component concentrations can then be utilized in the calculation of geothermometer readings at 306 to obtain a calculated temperature. For comparison with the calculated temperature of 306, workflow 300 can continue at 308 with measuring well log temperatures at various depths of the hydrocarbon well. The well logging temperatures obtained at 308 can represent a measured temperature to provide an accurate assessment of downhole thermal parameters and gradients.

Workflow 300 can include determining if the measured temperature matches the calculated temperature at 310, such that the discrepancies between the well log temperature and geothermometer can signify contamination in the formation water. Thus, if the measured and calculated temperatures are determined to be equal (within a margin of error) at 310, workflow 300 can continue at 312 with identifying the formation water as uncontaminated. The equivalent temperatures between the well logs and the geothermometer can signify that no additional thermal effects or particles preventing thermodynamic equilibrium are present in the formation water.

In contrast, if discrepancies are identified at 310 relating to the measured and calculated temperatures (outside of the margin of error), workflow 300 can continue at 314 with identifying the formation water as contaminated. Regardless of the formation water being pristine or contaminated, workflow 300 can continue at 316 with providing the identified contamination levels to operational equipment (e.g., hydrocarbon operation management tool 114 and/or hydrocarbon extraction equipment 210). The identified contamination at 316 can be used in adjusting or optimizing a variety of drilling and production operations. In some embodiments, workflow 300 can continue at 318 with adjusting or identifying drilling parameters, including drilling fluid weight, casing failure, and circulation loss within the hydrocarbon well. In further embodiments, workflow 300 can continue at 320 with an estimation of hydrocarbon-in-place values, when the formation water is determined to be pristine. In further embodiments still, workflow 300 can continue at 322 with quantifying and monitoring the propagation of injected seawater for enhanced oil recovery.

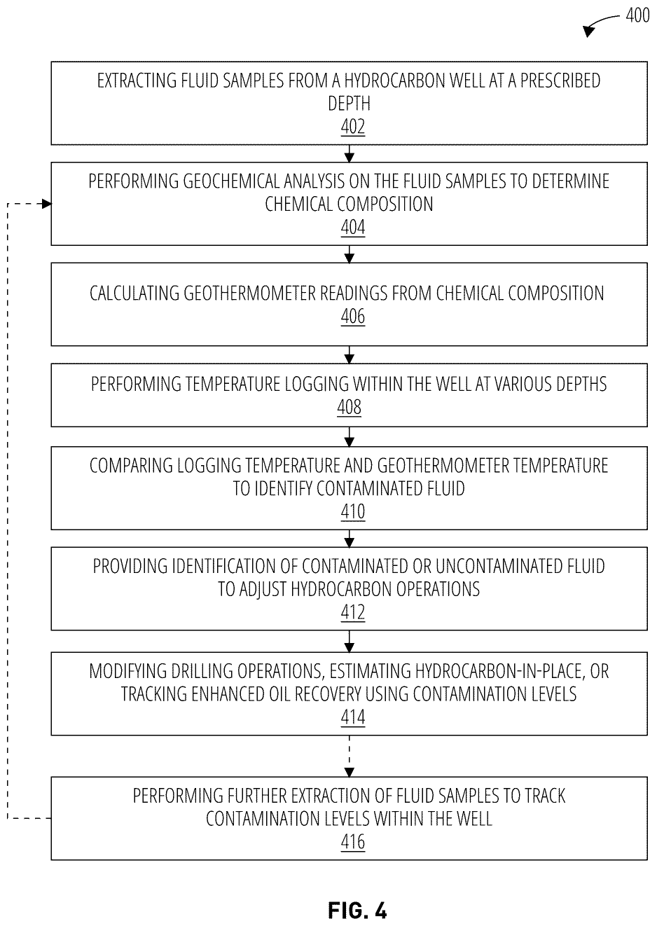

In view of the structural and functional features described above, example methods will be better appreciated with reference to FIG. 4. While, for purposes of simplicity of explanation, the example methods of FIG. 4 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement the methods, and conversely, some actions may be performed that are omitted from the description.

FIG. 4 illustrates a method 400 for identifying fluid contamination in a hydrocarbon well and modifying hydrocarbon extraction operations in response to the fluid contamination, according to one or more embodiments of the present disclosure. Method 400 can be implemented by system 100 and/or electronic device 200, as shown in FIGS. 1-2, and using workflow 300 of FIG. 3. As such, reference may be made to the examples of FIGS. 1-3 in the description of method 400. Method 400 can begin at 402 with extracting fluid samples (e.g., via fluid sampler 104) from a hydrocarbon well (e.g., hydrocarbon well 208) at a prescribed depth. In some embodiments, the fluid samples extracted at 402 can be taken from a plurality of depths within the hydrocarbon well, such that geothermal gradients can be further analyzed via geothermometry. Method 400 can continue at 404 with performing geochemical analysis (e.g., via geochemical analysis module 106) on the fluid samples to determine chemical composition that includes cationic component concentrations. The chemical composition of the formation water can thus be analyzed for a presence of silica, sodium, calcium, magnesium, and lithium for use in geothermometer methods.

Thus, method 400 can continue at 406 with calculating geothermometer readings (e.g., via geothermometric calculation module 108) from the chemical composition determined at 404. In some embodiments, the geothermometer method uses a magnesium-corrected sodium-potassium-calcium geothermometer, a magnesium lithium geothermometer, a sodium-lithium geothermometer, or a combination thereof. The geothermometer readings at 406 can be utilized to determine one or more calculated temperatures to be compared to live data. As such, method 400 can continue at 408 with performing temperature logging (e.g., via well logging tool 110) within the hydrocarbon well at various depths to obtain a measured temperature. In some embodiments, as discussed above, the temperature logging at 408 can include measuring the temperature at a plurality of depths, such that a geothermal gradient can be constructed for the wellbore based upon the measured temperature taken at each of the plurality of depths. Further, the measured temperature at the plurality of depths can be utilized alongside the calculated temperatures at the plurality of depths to identify possible zones of contamination or damage to the wellbore.

With the measured and calculated temperatures obtained, method 400 can continue at 410 with comparing the measured logging temperature and the calculated geothermometer temperature to identify contaminated or pristine formation water (e.g., via fluid contamination assessment module 112). In some embodiments, method 400 can include determining if the calculated temperature is within a margin of error of the measured temperature at 410, to thus determine if the results are significant enough to classify the formation water as contaminated. In these embodiments, if the measured and calculated temperatures are within the margin of error of each other, the formation water can be assumed to be pristine. However, if the difference between the measured and calculated temperatures are outside of the margin of error, the formation water can be assumed to be contaminated at 410.

Method 400 can continue at 412 with providing the identification of contaminated or pristine formation water to a tool (e.g., hydrocarbon operation management tool 114) for adjusting hydrocarbon operations within the wellbore. Based upon the identification of the formation water as either pristine or contaminated, the tool can perform a variety of modifications to hydrocarbon operations. Thus, method 400 can continue at 414 with modifying drilling operations, estimating hydrocarbon-in-place, or tracking enhanced oil recovery using contamination levels and tool, or any combination thereof. The modifications of hydrocarbon operations at 414 can include adjusting drilling fluid properties, correcting circulation loss, and correcting casing failure (e.g., via drilling operations module 116), assessing hydrocarbon-in-place estimates (e.g., via hydrocarbon-in-place module 118), adjusting seawater injection levels for enhanced oil recovery (e.g., via enhanced oil recovery module 120), or a combination thereof. The adjustments and modifications of the hydrocarbon operations at 414 can be further carried out via hydrocarbon extraction equipment (e.g., hydrocarbon extraction equipment 210), such that the tool of 412 can be in communication with active equipment within the hydrocarbon well.

In some embodiments, however, method 400 can continue at 416 with performing further extraction of fluid samples to track contamination levels within the hydrocarbon well. The further extraction performed at 416 can be utilized in the active monitoring of the hydrocarbon well, such that the timing and severity of contamination can be captured. Method 400 can accordingly return to 404 and continue cyclically as the calculated temperature of the further fluid sample is compared to a new measured temperature to continue monitoring for contamination.

### Examples

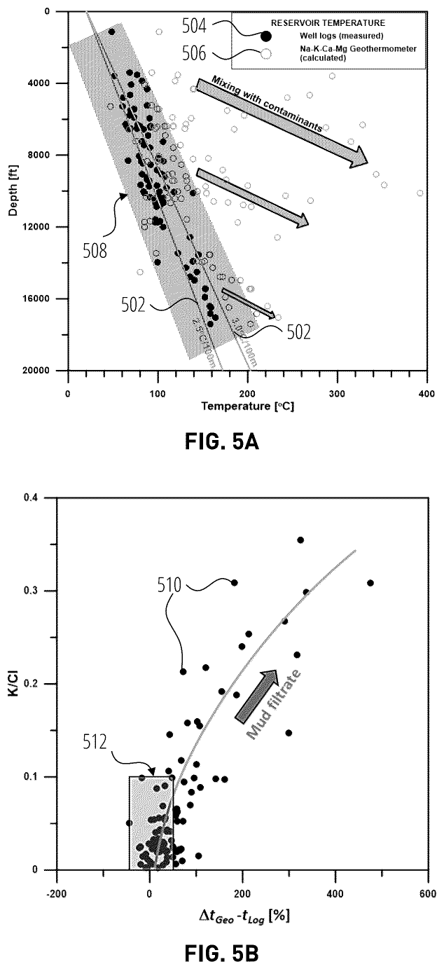

As a practical case study, geochemical data from 105 produced water samples was compiled to calculate reservoir temperatures with the Na—Ka—Ca—Mg geothermometers for each sample and to compare calculated t_{Na—K—Ca Mg corr} (t_{Geo}) with measured well log reservoir temperatures (t_{Log}). FIG. 5A depicts vertical trends of geothermal gradients 502 and measured reservoir temperatures 504 at specific depths, compared with geothermometer temperatures 506 from produced water. It can be seen that most of the temperatures are within the vertical trend of the average global geothermal gradient between 2.5 and 3 °C, although a slight homogenization trend seems to cause slightly elevated temperature values 504 for selected samples at a depth between 3,500 and 7,000 ft, and cooler conditions between 8,000 and 12,000 ft. In contrast, calculated geothermometer temperatures 506, reconstructed with the chemical composition of produced water, are strongly dispersed throughout the lithological column. t_{Geo} values within a range 508 of ±30 °C to measured well log temperatures are considered to reflect the dominance of formation water in the produced water sample. In contrast, increasing deviations of Na—K—Ca—Mg geothermometer temperatures from the natural geothermal gradient and from natural reservoir conditions suggest an enhanced contribution of contaminants in produced water. For the present exploration case study, the increasing discrepancy between calculated reservoir temperatures 506 and measured reservoir temperatures 504 (Δt_{Geo}−t_{log}) is positively correlated with growing potassium concentrations (reflected by increasing K/Cl ratios), as seen in FIG. 5B. FIG. 5B depicts the discrepancies 510 between calculated reservoir temperatures 506 and measured reservoir temperatures 504 and its increase with a growing K/Cl ratio, suggesting enhanced mixing with contaminant fluids. If the permitted discrepancy value 510 is limited to a maximum error range of +30%, a K/Cl ratio below 0.1 reflects the natural composition of formation water 512, while most samples with a ratio above 0.1 are affected by contaminant fluids, such as K-enriched mud filtrates.

### Example Computer System

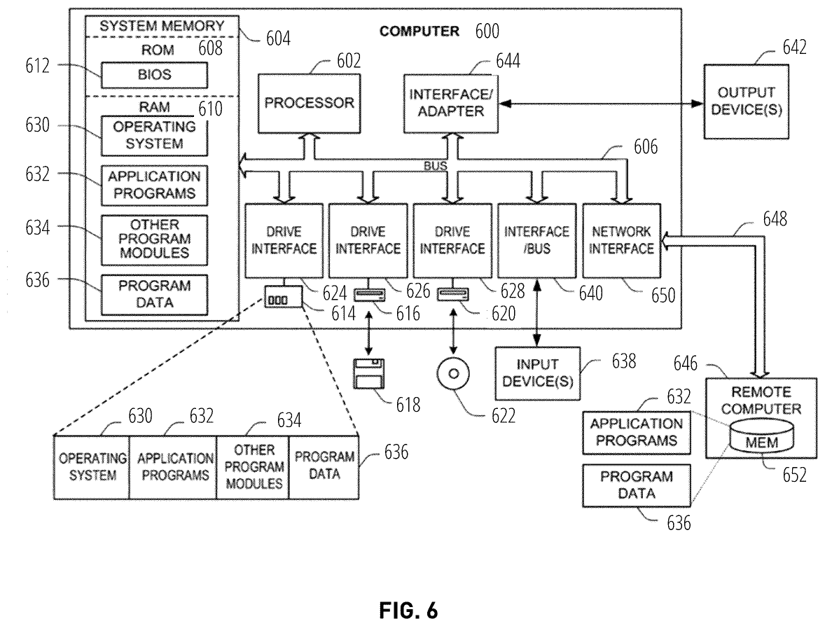

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software, firmware and/or hardware, such as shown and described with respect to the computer system of FIG. 6. Furthermore, portions of the embodiments may be a computer program product on a computer-readable storage medium having computer readable program code on the medium. Any non-transitory, tangible storage media possessing structure may be utilized including, but not limited to, static and dynamic storage devices, volatile and non-volatile memories, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general-purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to realize a computer implemented process such that the instructions which execute on the computer or other programmable apparatus provide steps for implementing the functions specified in flowchart blocks that may be described herein.

In this regard, FIG. 6 illustrates one example of a computer system 600 that can be employed to execute one or more embodiments of the present disclosure. Computer system 600 can be implemented on one or more general purpose networked computer systems, embedded computer systems, routers, switches, server devices, client devices, various intermediate devices/nodes, or standalone computer systems. Additionally, computer system 600 can be implemented on various mobile clients such as, for example, a personal digital assistant (PDA), laptop computer, pager, and the like, provided it includes sufficient processing capabilities.

Computer system 600 includes processing unit 602, system memory 604, and system bus 606 that couples various system components, including the system memory 604, to processing unit 602. System memory 604 can include volatile (e.g., RAM, DRAM, SDRAM, Double Data Rate (DDR) RAM, etc.) and non-volatile (e.g., Flash, NAND, etc.) memory. Dual microprocessors and other multi-processor architectures also can be used as processing unit 602. System bus 606 may be any of several types of bus structure including a memory bus or memory controller, a peripheral bus, and a local bus using any of a variety of bus architectures. System memory 604 includes read only memory (ROM) 608 and random-access memory (RAM) 610. A basic input/output system (BIOS) 612 can reside in ROM 608 containing the basic routines that help to transfer information among elements within computer system 600.

Computer system 600 can include a hard disk drive 614, magnetic disk drive 616, e.g., to read from or write to removable disk 618, and an optical disk drive 620, e.g., for reading CD-ROM disk 622 or to read from or write to other optical media. Hard disk drive 614, magnetic disk drive 616, and optical disk drive 620 are connected to system bus 606 by a hard disk drive interface 624, a magnetic disk drive interface 626, and an optical drive interface 628, respectively. The drives and associated computer-readable media provide nonvolatile storage of data, data structures, and computer-executable instructions for computer system 600. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and ROM 608, including operating system 630, one or more application programs 632, other program modules 634, and program data 636. In some examples, the application programs 632 can include fluid contamination determination engine 102, geochemical analysis module 106, geothermometric calculation module 108, fluid contamination assessment module 112, hydrocarbon operation management tool 114, drilling operations module 116, hydrocarbon-in-place module 118, and enhanced oil recovery module 120. The program data 636 can include any of the readings of fluid sampler 104 and well logging tool 110, the cationic component concentrations of the geochemical analysis module 106, the measured and calculated temperatures, and any combination thereof. The application programs 632 and program data 636 can include functions and methods programmed to identify contamination in formation water within a wellbore and modify hydrocarbon extraction operations based upon the contamination, such as shown and described herein.

A user may enter commands and information into computer system 600 through one or more input device 638, such as a pointing device (e.g., a mouse, touch screen), keyboard, microphone, joystick, game pad, scanner, and the like. These and other input devices 638 are often connected to processing unit 602 through a corresponding port interface 640 that is coupled to the system bus, but may be connected by other interfaces, such as a parallel port, serial port, or universal serial bus (USB). One or more output devices 642 (e.g., display, a monitor, printer, projector, or other type of displaying device) is also connected to system bus 606 via interface 644, such as a video adapter.

Computer system 600 may operate in a networked environment using logical connections to one or more remote computers, such as remote computer 646. Remote computer 646 may be a workstation, computer system, router, peer device, or other common network node, and typically includes many or all the elements described relative to computer system 600. The logical connections, schematically indicated at 648, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 600 can be connected to the local network through a network interface or adapter 650. When used in a WAN networking environment, computer system 600 can include a modem, or can be connected to a communications server on the LAN. The modem, which may be internal or external, can be connected to system bus 606 via an appropriate port interface. In a networked environment, application programs 632 or program data 636 depicted relative to computer system 600, or portions thereof, may be stored in a remote memory storage device 652.

In further embodiments, processing may be distributed among one or more processors at the same or different locations over a network. Workflow steps 302-322, processing steps 402-416 in method 400, fluid contamination determination engine 102 and its components (geochemical analysis module 106, geothermometric calculation module 108, fluid contamination assessment module 112), and hydrocarbon operation management tool 114 and its components (drilling operations module 116, hydrocarbon-in-place module 118, enhanced oil recovery module 120) can be implemented on any type of computing device including, but not limited to, a laptop, desktop, tablet, workstation, mobile device or smartphone, kiosk, embedded system, or other computing device having at least one processor and a non-transitory computable readable memory. The computing device may include a browser, application, and operating system along with a user-interface depending upon a desired configuration. The computing device may have functionality performed at the same or different physical locations and by one or more processors located at the same or different locations. A computing device may also be coupled to one or more application programming interfaces (APIs) to perform or distribute aspects of the functionality described herein. Computing functionality as described herein may also be implemented on a server, cluster of servers, web server, cloud-computing platform, and/or other remote service. A client/server architecture may also be implemented as would be apparent to a person skilled in the art given this description. In some embodiments, hydrocarbon operation management tool 114 may be provided on a remote server, accessed by an API call or other request, or otherwise in communication with fluid contamination determination engine 102 and the computing device thereof.

Embodiments disclosed herein include:
A. A computer-implemented method for identifying contamination in formation water includes extracting a fluid sample of formation water from a hydrocarbon well, performing geochemical analysis on the fluid sample to determine chemical composition of the formation water, and generating a calculated temperature using a geothermometer method and the chemical composition of the formation water. The computer-implemented method further includes performing temperature logging in the well at one or more depths to obtain a measured temperature, comparing the calculated temperature to the measured temperature to determine a presence of contamination within the formation water, and modifying hydrocarbon extraction operations in response to the presence of contamination within the formation water.
B. A system for identifying contamination in formation water includes a fluid sampler included at or near a wellbore of interest, the fluid sampler operable to extract a sample of formation water from a depth of the wellbore, a well logging tool included at or near the wellbore and operable to determine a measured temperature within the wellbore at the depth, and a fluid contamination determination engine in communication with the fluid sampler and the well logging tool and operable to detect contamination within the sample and the wellbore. The fluid contamination determination engine is configured for implementation on at least one processor and includes a geochemical analysis module operable to receive the sample of the formation water and perform a geochemical analysis extracting cationic component concentrations of the sample, a geothermometric calculation module operable to utilize the cationic component concentrations of the sample to generate a calculated temperature via one or more geothermometer methods, and a fluid contamination assessment module operable to compare the measured temperature and the calculated temperature and determine a presence of contamination within the sample of formation water.
C. A non-transitory computer-readable medium stores machine-readable instructions, which, when executed by a processor of an electronic device, cause the electronic device to receive a sample of formation water for a wellbore of interest from a fluid sampler in communication with the electronic device, perform geochemical analysis on the sample to determine cationic component concentrations in the sample, generate a calculated temperature for the sample using the cationic component concentrations and one or more geothermometer methods, receive a measured temperature for the wellbore from a well logging tool in communication with the electronic device, and compare the calculated temperature to the measured temperature to determine a presence of contamination within the formation water of the wellbore.

Each of embodiments A through C may have one or more of the following additional elements in any combination: Element 1: wherein the chemical composition of the formation water is analyzed for a presence of sodium, calcium, magnesium, and lithium for use in the geothermometer method. Element 2: wherein the geothermometer method uses a magnesium-corrected sodium-potassium-calcium geothermometer, a magnesium lithium geothermometer, a sodium-lithium geothermometer, or a combination thereof. Element 3: wherein modifying hydrocarbon operations includes adjusting drilling fluid properties, correcting circulation loss, correcting casing failure, assessing hydrocarbon-in-place estimates, adjusting seawater injection levels for enhanced oil recovery, or a combination thereof. Element 4: wherein comparing the calculated temperature to the measured temperature further comprises: determining if the calculated temperature is within a margin of error of the measured temperature; identifying the formation water as uncontaminated formation water in response to the calculated temperature being within the margin of error about the measured temperature; and identifying the formation water as contaminated formation water in response to the calculated temperature being outside of the margin of error about the measured temperature. Element 5: further comprising: extracting a further fluid sample from the hydrocarbon well after identifying the uncontaminated formation water; and comparing a calculated temperature of the further fluid sample to the measured temperature to monitor for contamination. Element 6: wherein temperature logging in the hydrocarbon well is performed at a plurality of depths, and wherein the computer-implemented method further comprises: constructing a geothermal gradient for the hydrocarbon well based upon the measured temperature taken at each of the plurality of depths.

Element 7: further comprising: a hydrocarbon operation management tool in communication with the fluid contamination determination engine to receive data regarding the presence of contamination within the sample of formation water and modify hydrocarbon operations within the wellbore. Element 8: wherein the hydrocarbon operation management tool includes a drilling operations module operable to generate instructions for adjusting drilling fluid properties, correcting circulation loss within the wellbore, repairing casing failures within the wellbore, or a combination thereof. Element 9: further comprising: hydrocarbon extraction equipment in communication with the hydrocarbon operation management tool and operable to receive and execute the instructions generated by the hydrocarbon operation management tool. Element 10: wherein the hydrocarbon operation management tool includes a hydrocarbon-in-place module operable to generate an estimate of hydrocarbon-in-place levels within the wellbore and generate instructions to modify extraction operations using the estimate. Element 11: wherein the hydrocarbon operation management tool includes an enhanced oil recovery module operable to monitor and control an injection of seawater in the wellbore for enhanced oil recovery based upon the presence of contamination within the sample of formation water. Element 12: wherein the fluid contamination determination engine and the hydrocarbon operation management tool are included in an electronic device in communication with the fluid sampler and the well logging tool.

Element 13: wherein the geothermometric calculation module utilizes a magnesium-corrected sodium-potassium-calcium geothermometer, a magnesium lithium geothermometer, a sodium-lithium geothermometer, or a combination thereof. Element 14: wherein the instructions, when executed by the processor of the electronic device, further cause the electronic device to: generate instructions to modify hydrocarbon extraction within the wellbore of interest based upon the presence of contamination within the formation water; and transmit said instructions to hydrocarbon extraction equipment within the wellbore for performance of said instructions. Element 15: wherein the instructions include an adjustment of drilling fluid composition, a repair of failed casing within the wellbore, a correction of a lost-circulation zone, or a combination thereof. Element 16: wherein the instructions, when executed by the processor of the electronic device, further cause the electronic device to: request a further sample of formation water from the fluid sampler; and compare a calculated temperature of the further sample to the measured temperature to monitor for contamination. Element 17: wherein the machine-readable instructions utilize a magnesium-corrected sodium-potassium-calcium geothermometer, a magnesium lithium geothermometer, a sodium-lithium geothermometer, or a combination thereof to generate the calculated temperature.

By way of non-limiting example, exemplary combinations applicable to A through C include: Element 1 with Element 2; Element 4 with Element 5; Element 7 with Element 8; Element 8 with Element 9; Element 7 with Element 10; Element 7 with Element 11; Element 7 with Element 12; and Element 14 with Element 15.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A computer-implemented method for identifying contamination in formation water, the method comprising:
extracting a fluid sample of formation water from a hydrocarbon well;
performing geochemical analysis on the fluid sample to determine chemical composition of the formation water;
generating a calculated temperature using a geothermometer method and the chemical composition of the formation water;
performing temperature logging in the well at one or more depths to obtain a measured temperature;
comparing the calculated temperature to the measured temperature to determine a presence of contamination within the formation water; and
modifying hydrocarbon extraction operations in response to the presence of contamination within the formation water.

2. The computer-implemented method of claim 1, wherein the chemical composition of the formation water is analyzed for a presence of sodium, calcium, magnesium, and lithium for use in the geothermometer method.

3. The computer-implemented method of claim 2, wherein the geothermometer method uses a magnesium-corrected sodium-potassium-calcium geothermometer, a magnesium lithium geothermometer, a sodium-lithium geothermometer, or a combination thereof.

4. The computer-implemented method of claim 1, wherein modifying hydrocarbon operations includes adjusting drilling fluid properties, correcting circulation loss, correcting casing failure, assessing hydrocarbon-in-place estimates, adjusting seawater injection levels for enhanced oil recovery, or a combination thereof.

5. The computer-implemented method of claim 1, wherein comparing the calculated temperature to the measured temperature further comprises:
determining if the calculated temperature is within a margin of error of the measured temperature;
identifying the formation water as uncontaminated formation water in response to the calculated temperature being within the margin of error about the measured temperature; and
identifying the formation water as contaminated formation water in response to the calculated temperature being outside of the margin of error about the measured temperature.

6. The computer-implemented method of claim 5, further comprising:
extracting a further fluid sample from the hydrocarbon well after identifying the uncontaminated formation water; and
comparing a calculated temperature of the further fluid sample to the measured temperature to monitor for contamination.

7. The computer-implemented method of claim 1, wherein temperature logging in the hydrocarbon well is performed at a plurality of depths, and wherein the computer-implemented method further comprises:
constructing a geothermal gradient for the hydrocarbon well based upon the measured temperature taken at each of the plurality of depths.

8. A system for identifying contamination in formation water, the system comprising:
a fluid sampler included at or near a wellbore of interest, the fluid sampler operable to extract a sample of formation water from a depth of the wellbore;
a well logging tool included at or near the wellbore and operable to determine a measured temperature within the wellbore at the depth; and
a fluid contamination determination engine in communication with the fluid sampler and the well logging tool and operable to detect contamination within the sample and the wellbore, wherein the fluid contamination determination engine is configured for implementation on at least one processor and includes:
a geochemical analysis module operable to receive the sample of the formation water and perform a geochemical analysis extracting cationic component concentrations of the sample,
a geothermometric calculation module operable to utilize the cationic component concentrations of the sample to generate a calculated temperature via one or more geothermometer methods, and
a fluid contamination assessment module operable to compare the measured temperature and the calculated temperature and determine a presence of contamination within the sample of formation water.

9. The system of claim 8, further comprising:
a hydrocarbon operation management tool in communication with the fluid contamination determination engine to receive data regarding the presence of contamination within the sample of formation water and modify hydrocarbon operations within the wellbore.

10. The system of claim 9, wherein the hydrocarbon operation management tool includes a drilling operations module operable to generate instructions for adjusting drilling fluid properties, correcting circulation loss within the wellbore, repairing casing failures within the wellbore, or a combination thereof.

11. The system of claim 10, further comprising:
hydrocarbon extraction equipment in communication with the hydrocarbon operation management tool and operable to receive and execute the instructions generated by the hydrocarbon operation management tool.

12. The system of claim 9, wherein the hydrocarbon operation management tool includes a hydrocarbon-in-place module operable to generate an estimate of hydrocarbon-in-place levels within the wellbore and generate instructions to modify extraction operations using the estimate.

13. The system of claim 9, wherein the hydrocarbon operation management tool includes an enhanced oil recovery module operable to monitor and control an injection of seawater in the wellbore for enhanced oil recovery based upon the presence of contamination within the sample of formation water.

14. The system of claim 9, wherein the fluid contamination determination engine and the hydrocarbon operation management tool are included in an electronic device in communication with the fluid sampler and the well logging tool.

15. The system of claim 8, wherein the geothermometric calculation module utilizes a magnesium-corrected sodium-potassium-calcium geothermometer, a magnesium lithium geothermometer, a sodium-lithium geothermometer, or a combination thereof.

16. A non-transitory computer-readable medium storing machine-readable instructions, which, when executed by a processor of an electronic device, cause the electronic device to:
receive a sample of formation water for a wellbore of interest from a fluid sampler in communication with the electronic device;
perform geochemical analysis on the sample to determine cationic component concentrations in the sample;
generate a calculated temperature for the sample using the cationic component concentrations and one or more geothermometer methods;
receive a measured temperature for the wellbore from a well logging tool in communication with the electronic device; and
compare the calculated temperature to the measured temperature to determine a presence of contamination within the formation water of the wellbore.

17. The non-transitory computer-readable medium of claim 16, wherein the instructions, when executed by the processor of the electronic device, further cause the electronic device to:
generate instructions to modify hydrocarbon extraction within the wellbore of interest based upon the presence of contamination within the formation water; and
transmit said instructions to hydrocarbon extraction equipment within the wellbore for performance of said instructions.

18. The non-transitory computer-readable medium of claim 17, wherein the instructions include an adjustment of drilling fluid composition, a repair of failed casing within the wellbore, a correction of a lost-circulation zone, or a combination thereof.

19. The non-transitory computer-readable medium of claim 16, wherein the instructions, when executed by the processor of the electronic device, further cause the electronic device to:
request a further sample of formation water from the fluid sampler; and
compare a calculated temperature of the further sample to the measured temperature to monitor for contamination.

20. The non-transitory computer-readable medium of claim 16, wherein the machine-readable instructions utilize a magnesium-corrected sodium-potassium-calcium geothermometer, a magnesium lithium geothermometer, a sodium-lithium geothermometer, or a combination thereof to generate the calculated temperature.
